(19) United States
(12) Patent Application Publication
(10) Pub. No.: US 2020/0288843 A1
(43) Pub. Date: Sep. 17, 2020

(54) Device for Treating Human Skin, Hair and Nails

(71) Applicant: Johann Verheem, Weston, CT (US)

(72) Inventor: Johann Verheem, Weston, CT (US)

(21) Appl. No.: 16/819,016

(22) Filed: Mar. 13, 2020

Related U.S. Application Data
(60) Provisional application No. 62/878,150, filed on Jul. 24, 2019.
(60) Provisional application No. 62/818,180, filed on Mar. 14, 2019.

(51) Int. Cl. A45D 34/04; A61N 5/06

(52) U.S. Cl. CPC A45D 34/042; A45D 2200/205; A61N 2005/0651; A61N 5/0616

(57) ABSTRACT

A device for applying soluble ingredient to a user's skin, scalp, hair or nails that simultaneously dispenses the soluble ingredient and therapeutic light.

### CROSS-REFERENCE TO RELATED APPLICATIONS

This application is a United States non-provisional application which is related to U.S. provisional patent application No. 62/818,180 entitled “Device for cleaning and applying skin care ingredients” and filed Mar. 14, 2019, as well as U.S. provisional patent application No. 62/878,150 entitled “Device for treating skin” and filed on Jul. 24, 2019, the contents of which is incorporated herein by reference in its entirety as if set forth verbatim.

### FIELD

The following description relates generally to a device that is used apply ingredients to human skin, hair and nails.

### BACKGROUND

Skin care is a common field and many different types of skin creams, lotions, soaps and medications are applied to the skin in order to treat or prevent aging, affect wrinkles, reduce discoloration, pigmentation and age spots and eliminate medical skin conditions such as acne. Skin care and hair care can also include treatment of the human scalp. In addition, nails are often treated with creams, lotions and decorated with paints and other soluble ingredients.

Phototherapy and the use of various different light sources and frequencies, including laser light and infrared light have also been used to provide cosmetic and therapeutic benefits to human skin, hair and nails. The current invention consists of a device that combines a source of light and phototherapy with the ability to dispense soluble ingredients that provides therapeutic of cosmetic benefits to the user's skin, hair or nails.

### SUMMARY

The following simplified summary is provided in order to provide a basic understanding of some aspects of the claimed subject matter. This summary is not an extensive overview and is not intended to identify key/critical elements or to delineate the scope of the claimed subject matter. Its purpose is to present some concepts in a simplified form as a prelude to the more detailed description that is presented later.

The current invention provides a novel device that combines a container that dispenses a soluble ingredient while at the same time providing light or therapeutic light that compliments or enhances the treatment of the part of the skin, hair or nails of the user to which the ingredient is being applied.

To the accomplishment of the foregoing and related ends, certain illustrative aspects are described herein in connection with the following description and the annexed drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE DRAWINGS

FIG. 1 provides a view of a squeezable tube with an attached applicator that contains a cavity through which the ingredient in the tube can be dispensed (A) as well as one or more sources of light (B)

FIG. 2 provides and additional view of a tube that contains an ingredient (A) and an applicator cap (C) that also contains one of more light sources (B) that are attached to the applicator cap.

FIG. 3 is a perspective view of a rigid or semi rigid tube with an applicator cap attached on one end, in which one or more light sources (A) are embedded or attached to, as well as a rotatable activator (B) that will squeeze the ingredient embedded in the rigid or semi right tube out through the applicator.

FIG. 4 shows a perspective view of an embodiment of the device where the applicator is a rotating brush that moves (B) where the ingredient is housed within the body of the brush (C) and where the light source is embedded in between the bristles of the device (A)

### DETAILED DESCRIPTION

The features of the presently disclosed solution may be economically molded or assembled by using one or more distinct parts and associated components which, may be assembled together for removable or integral application. Unless defined otherwise, all terms of art, notations and other scientific terms or terminology used herein have the same meaning as is commonly understood by one of ordinary skill in the art to which this disclosure belongs.

In some cases, terms with commonly understood meanings are defined herein for clarity and/or for ready reference, and the inclusion of such definitions herein should not necessarily be construed to represent a substantial difference over what is generally understood in the art. All patents, applications, published applications and other publications referred to herein are incorporated by reference in their entirety. If a definition set forth in this section is contrary to or otherwise inconsistent with a definition set forth in the patents, application, published applications and other publications that are herein incorporated by reference, the definition set forth in this section prevails over the definition that is incorporated herein by reference.

As used herein, “a” or “an” means “at least one” or “one or more.” As used herein, the term “user”, “subject”, “end-user” or the like is not limited to a specific entity or person. For example, the term “user” may refer to a person who uses the systems and methods described herein, and frequently may be a technician. However, this term is not limited to end users or technicians and thus encompasses a variety of persons or entities who can use the disclosed systems and methods.

As used herein, the term applicator device is used herein with reference to a device that combines a container that dispenses a soluble ingredient while at the same time providing light or therapeutic light that compliments or enhances the treatment of the part of the skin, hair or nails of the user to which the ingredient is being applied.

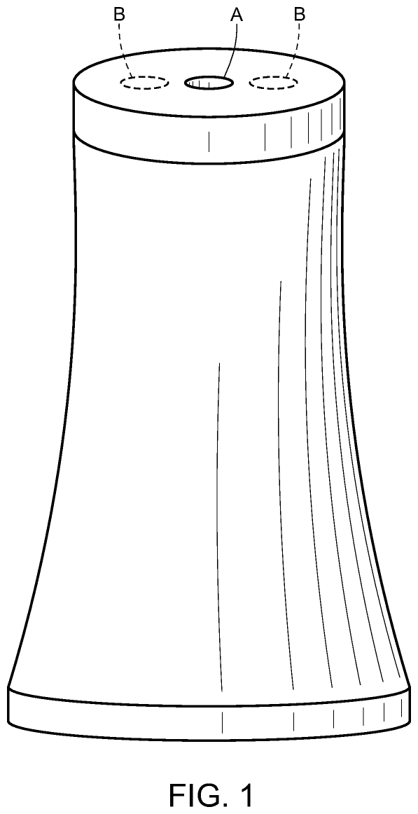

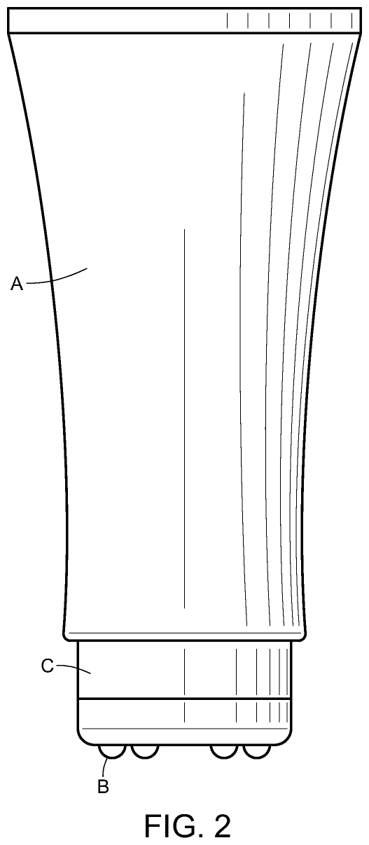

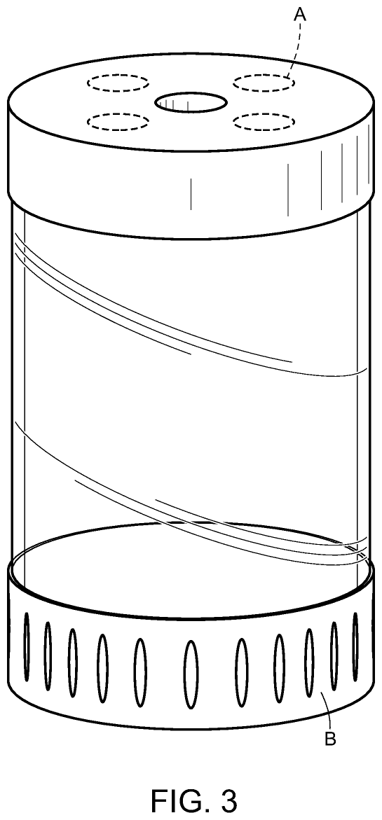

The disclosed solution can now be better understood turning to the following detailed description. It is to be expressly understood that the illustrated embodiments are set forth as examples and not by way of limitations on the embodiments as ultimately defined in the claims. Specifically, FIGS. 1-4 show various exemplary embodiments of the herein described improved charging devices for use with a watch.

In one aspect of the disclosed embodiments, an applicator device may consist of a tube that contains an ingredient with an applicator that contains a power source and a light source that is attached to the tube. FIG. 1 demonstrates a tube that is attached to an applicator through which the ingredient inside the tube will be squeezed out, while the applicator also contains a light source, such as a laser of LED light. When the user presses the applicator against their skin, hair, nails or scalp and squeezes out the ingredients inside the tube, the ingredient penetrates or bonds with the user's skin, hair, scalp or nails. Simultaneously, the light source reaches the ingredient as well as the user's skin, hair, scalp or nails.

The light source can impact the ingredient, so that chemical processes or alterations in the ingredient is activated. The light source can also impact the user's skin, scalp or nails, by increasing blood flow, in the case of certain red frequencies of light, or by killing bacteria or viruses on the surface of the skin, such is the case in with certain blue frequencies of light.

The light source can also act as a source of heat that melts the ingredient when dispensed through the applicator or that heats up either the ingredient, the skin, hair or nails of the user or both ingredient and body part of the user in order to provide therapeutic or cosmetic benefits.

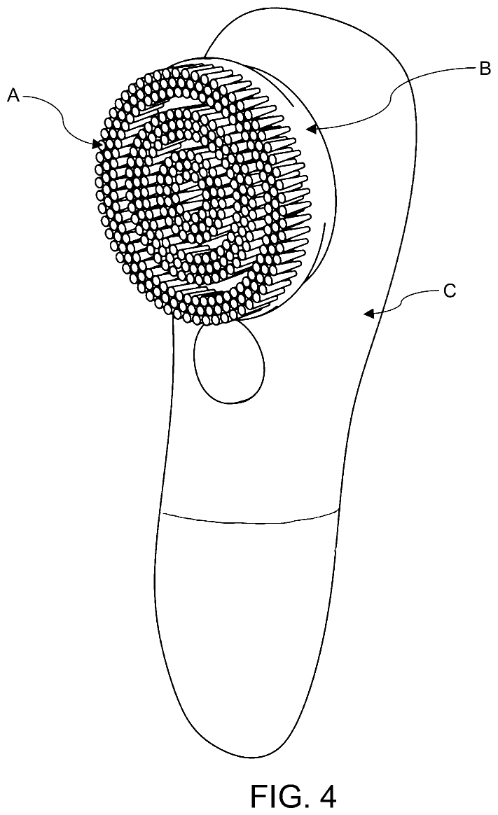

In one embodiment of the device, the applicator can be a rotating brush or applicator surface that contains one or more sources of light and that project on the user's skin, hair or nails while the rotating brush or applicator surface is engages to clean, massage or provide therapeutic benefits to the user's skin, scalp, nails or hair. FIG. 4 demonstrates an applicator brush where the applicator can rotate, while simultaneously dispensing light and ingredients to the user's skin, scalp, hair or nails.

The definitions of the words or elements of the following claims are, therefore, defined in this specification to not only include the combination of elements which are literally set forth. It is also contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination(s).

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements. The claims are thus to be understood to include what is specifically illustrated and described above, what is conceptually equivalent, what can be obviously substituted and also what incorporates the essential idea of the embodiments.

What has been described above includes examples of one or more embodiments. It is, of course, not possible to describe every conceivable combination of components or methodologies for purposes of describing the aforementioned embodiments, but one of ordinary skill in the art may recognize that many further combinations and permutations of various embodiments are possible. Accordingly, the described embodiments are intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A device that contains a liquid, cream or other soluble ingredient that is dispensed on the skin, scalp, hair or nails of the user where the ingredient is dispensed while the device simultaneously emits therapeutic light on the skin, scalp, hair or nails of the user.

2. A device that contains a liquid, cream or other soluble ingredient that is dispensed on the skin, scalp, hair or nails of the user where the ingredient is dispensed through and applicator that it attached to the device, while the device simultaneously emits therapeutic light on the skin, scalp, hair or nails of the user.

3. A device with an applicator that emits light and dispenses an ingredient contained within the device to the skin, where such therapeutic light is only activated when the user presses the device against their skin, hair, scalp or nails.

4. A device as described in claim 2, where the frequency, color or temperature of the light interacts with the ingredient in a manner that improves the efficacy of the ingredient's therapeutic, cosmetic, phycological or medical benefits.

5. A device as described in claim 2, where the applicator rotates.

6. A device as described in claim 3, where the applicator rotates.

7. A device as described in claim 2 where the light source is mounted behind the a transparent or translucent applicator or brush, allowing the light to reach the user's skin, hair, scalp or nails by shining through the applicator or brush.

8. A device as described in claim 3 where the light source is mounted behind a transparent or translucent applicator or brush, allowing the light to reach the user's skin, hair, scalp or nails by shining through the applicator or brush.

9. A device as described in claim 4 where the light source is mounted behind a transparent or translucent applicator or brush, allowing the light to reach the user's skin, hair, scalp or nails by shining through the applicator or brush.

10. A device as described in claim 2 where the light source is mounted behind a applicator or brush with holes or cavities in the applicator or brush, allowing the light to reach the user's skin, hair, scalp or nails by shining through the holes or cavities in the applicator or brush.

11. A device as described in claim 3 where the light source is mounted behind a applicator or brush with holes or cavities in the applicator or brush, allowing the light to reach the user's skin, hair, scalp or nails by shining through the holes or cavities in the applicator or brush.

12. A device as described in claim 4 where the light source is mounted behind a applicator or brush with holes or cavities in the applicator or brush, allowing the light to reach the user's skin, hair, scalp or nails by shining through the holes or cavities in the applicator or brush.

13. A device as described in claim 3, that contains a clear or translucent rotating applicator or surface used to apply pressure on the skin for cleansing, treatment or massage where there are one or more light sources mounted behind the applicator of surface so that light shines through the applicator or surface.

14. A device as described in claim 3, that contains a rotating applicator or surface used to apply pressure on the skin for cleansing, treatment or massage where there are one or more light sources mounted within the applicator's surface so that light shines through the applicator or surface.
